(19) United States
(12) Patent Application Publication
(10) Pub. No.: US 2006/0084682 A1
(43) Pub. Date: Apr. 20, 2006

(54) Thrombopoietin mimetics

(76) Inventors: Dirk A Heerding, Collegeville, PA (US); Alan T. Price, Collegeville, PA (US); Igor Safonov, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/538,252

(22) PCT Filed: Dec. 12, 2003
(86) PCT No.: PCT/US03/39633

§ 371 Date: Jun. 9, 2005

Related U.S. Application Data
(60) Provisional application No. 60/433,482, filed on Dec. 13, 2002.

(51) Int. Cl. A61K 31/4439; A61K 31/427; C07D 417/14; C07D 417/02

(52) U.S. Cl. 514/338; 514/369; 546/269.7; 548/181

(57) ABSTRACT

Invented are non-peptide TPO mimetics. Also invented are novel processes and intermediates used in the preparation of the presently invented compounds. Also invented is a method of treating thrombocytopenia, in a mammal, including a human, in need thereof which comprises administering to such mammal an effective amount of a selected benzimidazole derivative.

### FIELD OF THE INVENTION

This invention relates to thrombopoietin (TPO) mimetics and their use as promoters of thrombopoiesis and megakaryocytopoiesis.

### BACKGROUND OF THE INVENTION

Megakaryocytes are bone marrow-derived cells, which are responsible for producing circulating blood-platelets. Although comprising <0.25% of the bone marrow cells in most species, they have >10 times the volume of typical marrow cells. See Kuter et al. Proc. Natl. Acad. Aci. USA 91: 11104-11108 (1994). Megakaryocytes undergo a process known as endomitosis whereby they replicate their nuclei but fail to undergo cell division and thereby give rise to polyploid cells. In response to a decreased platelet count, the endomitotic rate increases, higher ploidy megakaryocytes are formed, and the number of megakaryocytes may increase up to 3-fold. See Harker J. Clin. Invest. 47: 458-465 (1968). In contrast, in response to an elevated platelet count, the endomitotic rate decreases, lower ploidy megakaryocytes are formed, and the number of megakaryocytes may decrease by 50%.

The exact physiological feedback mechanism by which the mass of circulating platelets regulates the endomitotic rate and number of bone marrow megakaryocytes is not known. The circulating thrombopoietic factor involved in mediating this feedback loop is now thought to be thrombopoietin (TPO). More specifically, TPO has been shown to be the main humoral regulator in situations involving thrombocytopenia. See, e.g., Metcalf Nature 369:519-520 (1994). TPO has been shown in several studies to increase platelet counts, increase platelet size, and increase isotope incorporation into platelets of recipient animals. Specifically, TPO is thought to affect megakaryocytopoiesis in several ways: (1) it produces increases in megakaryocyte size and number; (2) it produces an increase in DNA content, in the form of polyploidy, in megakaryocytes; (3) it increases megakaryocyte endomitosis; (4) it produces increased maturation of megakaryocytes; and (5) it produces an increase in the percentage of precursor cells, in the form of small acetylcholinesterase-positive cells, in the bone marrow.

Because platelets (thrombocytes) are necessary for blood clotting and when their numbers are very low a patient is at risk of death from catastrophic hemorrhage, TPO has potential useful application in both the diagnosis and the treatment of various hematological disorders, for example, diseases primarily due to platelet defects (see Harker et al. Blood 91: 4427-4433 (1998)). Ongoing clinical trials with TPO have indicated that TPO can be administered safely to patients (see Basser et al. Blood 89: 3118-3128 (1997); Fanucchi et al. New Engl. J. Med. 336: 404-409 (1997)). In addition, recent studies have provided a basis for the projection of efficacy of TPO therapy in the treatment of thrombocytopenia, and particularly thrombocytopenia resulting from chemotherapy, radiation therapy, or bone marrow transplantation as treatment for cancer or lymphoma. (See Harker, Curr. Opin. Hematol. 6: 127-134 (1999)).

The gene encoding TPO has been cloned and characterized. See Kuter et al., Proc. Natl. Acad. Sci. USA 91: 11104-11108 (1994); Barley et al., Cell 77: 1117-1124 (1994); Kaushansky et al., Nature 369:568-571 (1994); Wendling et al., Nature 369: 571-574 (1994); and Sauvage et al., Nature 369: 533-538 (1994). Thrombopoietin is a glycoprotein with at least two forms, with apparent molecular masses of 25 kDa and 31 kDa, with a common N-terminal amino acid; sequence. See, Baatout, Haemostasis 27: 1-8 (1997); Kaushansky, New Engl. J. Med. 339: 746-754 (1998). Thrombopoietin appears to have two distinct regions separated by a potential Arg-Arg cleavage site. The amino-terminal region is highly conserved in man and mouse, and has some homology with erythropoietin and interferon-a and interferon-b. The carboxy-terminal region shows wide species divergence.

The DNA sequences and encoded peptide sequences for human TPO receptor (TPO-R; also known as c-mpl) have been described. (See, Vigon et al. Proc. Natl. Acad. Sci. USA 89: 5640-5644 (1992)). TPO-R is a member of the haematopoietin growth factor receptor family, a family characterized by a common structural design of the extracellular domain, including for conserved C residues in the N-terminal portion and a WSXWS motif close to the transmembrane region. (See Bazan Proc. Natl. Acad. Sci. USA 87: 6934-6938 (1990)). Evidence that this receptor plays a functional role in hematopoiesis includes observations that its expression is restricted to spleen, bone marrow, or fetal liver in mice (see Souyri et al. Cell 63: 1137-1147 (1990)) and to megakaryocytes, platelets, and CD34⁺ cells in humans (see Methia et al. Blood 82: 1395-1401 (1993)). Further evidence for TPO-R as a key regulator of megakaryopoiesis is the fact that exposure of CD34⁺ cells to synthetic oligonucleotides antisense to TPO-R RNA significantly inhibits the appearance of megakaryocyte colonies without affecting erythroid or myeloid colony formation. Some workers postulate that the receptor functions as a homodimer, similar to the situation with the receptors for G-CSF and erythropoietin. (see Alexander et al. EMBO J. 14: 5569-5578 (1995)).

The slow recovery of platelet levels in patients suffering from thrombocytopenia is a serious problem, and has lent urgency to the search for a blood growth factor agonist able to accelerate platelet regeneration (see Kuter, Seminars in Hematology, 37: Supp 4: 41-49 (2000)).

It would be desirable to provide compounds which allow for the treatment of thrombocytopenia by acting as a TPO mimetic.

As disclosed herein it has unexpectedly been discovered that certain benzimidazoles are effective as agonists of the TPO receptor, they are potent TPO mimetics.

### SUMMARY OF THE INVENTION

This invention relates to compounds of Formula (I): 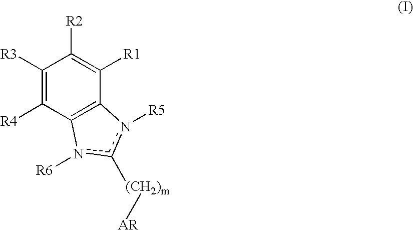
wherein:
the B ring has one double bond where indicated by the broken lines, provided that R⁵ is absent when the nitrogen attached thereto has a double bond and provided that R⁶ is absent when the nitrogen attached thereto has a double bond;
R¹, R², R³ and R⁴ are each independently selected from hydrogen, —(CH2)ₚOR¹⁰, —C(O)OR¹⁰, formyl, nitro, cyano, halogen, aryl, substituted aryl, substituted alkyl, —S(O)ₙR¹⁰, cycloalkyl, —NR¹¹R¹², protected —OH, —CONR¹¹R¹², phosphonic acid, sulfonic acid, phosphinic acid, —SO₂NR¹¹R¹², a heterocyclic methylene substituent as represented by Formula (II), 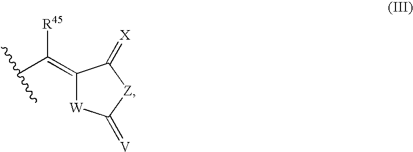
   and
a substituent as represented by Formula (VII), 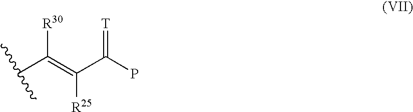
where,
p is 0-6,
n is 0-2,
W and Z are each independently selected from C, O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
V and X are each independently selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R¹⁰ is selected from: hydrogen, alkyl, cycloalkyl, C₁-C₁₂aryl, substituted alkyl, substituted cycloalkyl and substituted C₁-C₁₂aryl,
R¹¹ and R¹² are each independently selected from hydrogen, alkyl, substituted alkyl, C₃₋₆cycloalkyl, and aryl,
   or R¹¹ and R¹² taken together with the nitrogen to which they are attached represent a 5 to 6 member saturated ring containing up to one other heteroatom selected from oxygen and nitrogen,
T is absent or selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
P is selected from OR¹⁰, SR¹⁰, NR¹¹R¹², and R¹⁰, where R¹⁰ is selected from: hydrogen, alkyl, cycloalkyl, C₁-C₁₂aryl, substituted alkyl, substituted cycloalkyl and substituted C₁-C₁₂aryl,
R²⁵ is selected from: hydrogen, alkyl, cycloalkyl, C₁-C₁₂aryl, substituted alkyl, substituted cycloalkyl and substituted C₁-C₁₂aryl,
R³⁰ is selected from: hydrogen, alkyl, halogen, cycloalkyl, C₁-C₁₂aryl, substituted alkyl, substituted cycloalkyl and substituted C₁-C₁₂aryl, and
R⁴⁵ is selected from: hydrogen, alkyl, halogen, cycloalkyl, C₁-C₁₂aryl, substituted alkyl, substituted cycloalkyl and substituted C₁-C₁₂aryl;
R⁵ is absent when the nitrogen attached thereto has a double bond or selected from the group consisting of: hydrogen, alkyl, cycloalkyl, C₁-C₁₂aryl, substituted alkyl, substituted cycloalkyl and substituted C₁-C₁₂aryl;
R⁶ is absent when the nitrogen attached thereto has a double bond or selected from the group consisting of: hydrogen, alkyl, cycloalkyl, C₁-C₁₂aryl, substituted alkyl, substituted cycloalkyl and substituted C₁-C₁₂aryl;
m is 0-6; and
AR is a cyclic or polycyclic aromatic ring containing from 3 to 16 carbon atoms, optionally containing one or more heteroatoms, provided that when the number of carbon atoms is 3 the aromatic ring contains at least two heteroatoms and when the number of carbon atoms is 4 the aromatic ring contains at least one heteroatom, optionally substituted with one or more substituents selected from the group consisting of: alkyl, substituted alkyl, cycloalkyl, substituted cycloalkyl, aryl, substituted aryl, aryloxy, hydroxy, alkoxy, acyloxy, —NR¹³R¹⁴, N-acylamino, N-sulfonylamino, nitro, cyano, halogen, —C(O)OR¹⁰, —C(O)NR¹³R¹⁴, —S(O)₂NR¹³R¹⁴, —S(O)ₙR¹⁰, protected —OH, and alkyl substituted with one or more substituents selected from the group consisting of: alkoxy, acyloxy, aryl, substituted aryl, amino, N-acylamino, oxo, hydroxy, cycloalkyl, substituted cycloalkyl, —C(O)OR¹⁰, —S(O)₂NR¹³R¹⁴, —S(O)ₙR¹⁰, aryloxy, nitro, cyano, halogen, and protected —OH,
where
n is 0 to 2;
R¹⁰ is selected from the group consisting of: hydrogen, alkyl, cycloalkyl, C₁-C₁₂aryl, substituted alkyl, substituted cycloalkyl and substituted C₁-C₁₂aryl, and
R¹² and R¹³ are independently selected from the group consisting of: hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, —NR¹⁰R¹⁰, N-acylamino, oxo, hydroxy, —C(O)OR¹⁰, —S(O)ₙR¹⁰, —C(O)NR¹¹R¹⁰, —S(O)₂NR¹⁰R¹⁰, nitro, cyano, cycloalkyl, substituted cycloalkyl, halogen, C₁-C₁₂aryl, substituted C₁-C₁₂aryl, and protected —OH,
where n and R¹⁰ are as described above;
and pharmaceutically acceptable salts, hydrates, solvates and esters thereof.

This invention relates to a method of treating thrombocytopenia, which comprises administering to a subject in need thereof an effective amount of a TPO mimetic compound of Formula (I).

The present invention also relates to the discovery that the compounds of Formula (I) are active as agonists of the TPO receptor.

In a further aspect of the invention there is provided novel processes and novel intermediates useful in preparing the presently invented TPO mimetic compounds.

Included in the present invention are pharmaceutical compositions comprising a pharmaceutical carrier and compounds useful in the methods of the invention.

Also included in the present invention are methods of co-administering the presently invented TPO mimetic compounds with further active ingredients.

### DETAILED DESCRIPTION OF THE INVENTION

This invention relates to compounds of Formula (I) as described above.

Included among the presently invented compounds of Formula (I) are those having Formula (II): 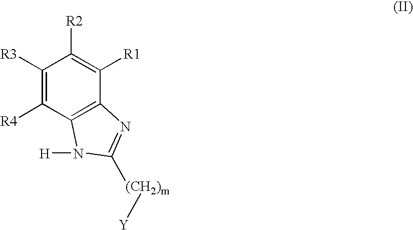
wherein:
R¹, R², R³ and R⁴ are each independently selected from hydrogen, —(CH₂)ₚOR¹⁰, —C(O)OR¹⁰, formyl, nitro, cyano, halogen, aryl, substituted aryl, substituted alkyl, —S(O)ₙR¹⁰, cycloalkyl, —NR¹¹R¹², protected —OH, —CONR¹¹R¹², phosphonic acid, sulfonic acid, phosphinic acid, —SO₂NR¹¹R¹², a heterocyclic methylene substituent as represented by Formula (III), 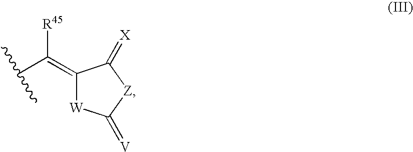
   and
a substituent as represented by Formula (VII), 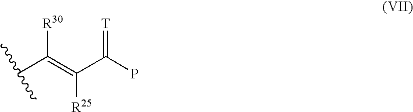
where,
p is 0-6,
n is 0-2,
W and Z are each independently selected from C, O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
V and X are each independently selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R¹⁰ is selected from: hydrogen, alkyl, cycloalkyl, C₁-C₁₂aryl, substituted alkyl, substituted cycloalkyl and substituted C₁-C₁₂aryl,
R¹¹ and R¹² are each independently selected from hydrogen, alkyl, substituted alkyl, C₃₋₆cycloalkyl, and aryl,
   or R¹¹ and R¹² taken together with the nitrogen to which they are attached represent a 5 to 6 member saturated ring containing up to one other heteroatom selected from oxygen and nitrogen,
T is absent or selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
P is selected from OR¹⁰, SR¹⁰, NR¹¹R¹², and R¹⁰, where R¹⁰ is selected from: hydrogen, alkyl, cycloalkyl, C₁-C₁₂aryl, substituted alkyl, substituted cycloalkyl and substituted C₁-C₁₂aryl,
R²⁵ is selected from: hydrogen, alkyl, cycloalkyl, C₁-C₁₂aryl, substituted alkyl, substituted cycloalkyl and substituted C₁-C₁₂aryl,
R³⁰ is selected from: hydrogen, alkyl, halogen, cycloalkyl, C₁-C₁₂aryl, substituted alkyl, substituted cycloalkyl and substituted C₁-C₁₂aryl, and
R⁴⁵ is selected from: hydrogen, alkyl, halogen, cycloalkyl, C₁-C₁₂aryl, substituted alkyl, substituted cycloalkyl and substituted C₁-C₁₂aryl;
m is 0-6and
Y is a cyclic or polycyclic aromatic ring containing from 4 to 14 carbon atoms, optionally containing from one to three heteroatoms, and optionally substituted with one or more substituents selected from the group consisting of: alkyl, substituted alkyl, C₁-C₁₂aryl, substituted cycloalkyl, substituted C₁-C₁₂aryl, hydroxy, aryloxy, alkoxy, cycloalkyl, nitro, cyano, halogen and protected —OH;
and pharmaceutically acceptable salts, hydrates, solvates and esters thereof.

Included among the presently invented compounds of Formula (II) are those in which R¹, R², R³ and R⁴ are each independently selected from hydrogen, C₁₋₆alkyl, hydroxy, nitro, cyano, halogen, C₁-C₁₂aryl, substituted C₁-C₁₂aryl, cycloalkyl, carboxylic acid, phosphonic acid, sulfonic acid, phosphinic acid, a substituent represented by Formula (III), 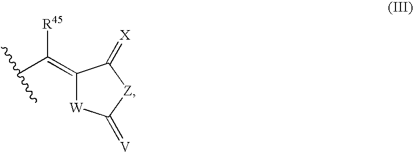
and
a substituent as represented by Formula (VI), 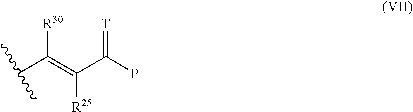
where,
W and Z are each independently selected from C, O, S and NR³⁵, where R³⁵ is selected from: hydrogen, alkyl, substituted alkyl, cycloallyl and C₁-C₁₂aryl,
V and X are each independently selected from O and S,
R¹⁰ is selected from: hydrogen, alkyl, cycloalkyl, C₁-C₁₂aryl, substituted alkyl, substituted cycloalkyl and substituted C₁-C₁₂aryl,
T is absent or selected from O, S and NR³⁵, where R³⁵ is selected from: hydrogen, alkyl, cycloalkyl, C₁-C₁₂aryl and substituted alkyl,
P is selected from OR⁴⁰, SR⁴⁰ and R⁴⁰, where R⁴⁰ is selected from: hydrogen, alkyl, cycloalkyl, C₁-C₁₂aryl and substituted alkyl,
R²⁵ is selected from: hydrogen, alkyl and substituted alkyl,
R³⁰ is selected from: hydrogen, alkyl, halogen and substituted alkyl, and
R⁴⁵ is selected from: hydrogen, alkyl, halogen, cycloalkyl, C₁-C₁₂aryl, substituted alkyl, substituted cycloalkyl and substituted C₁-C₁₂aryl;
m is 0; and
Y is selected from; phenyl, pyridyl, thiophene, piperidine, morpholine, thiomorpholine, 2-imidazoline and 1,3-dioxolane, where the phenyl, pyridyl, thiophene, piperidine, morpholine, thiomorpholine, 2-imidazoline and 1,3-dioxolane are optionally substituted with from one to three substituents selected from the group consisting of: alkyl, substituted alkyl, C₁-C₁₂aryl, substituted C₁-C₁₂aryl, alkoxy, hydroxy and halogen;
and pharmaceutically acceptable salts, hydrates, solvates and esters thereof.

Preferred among the presently invented compounds are:
5-{2-[6-(3,4-Dichloro-phenyl)-pyridin-2-yl]-1H-benzoimidazol-5-ylmethylene}-2-thioxo-thiozolidin-4-one;
5-{2-[6-(3,4-Dimethyl-phenyl)-pyridin-2-yl]-1H-benzoimidazol-5-ylmethylene}-2-thioxo-thiozolidin-4-one;
(E)-3-{2-[6-(4-tert-Butyl-phenyl)-pyridin-2-yl]-1H-benzoimidazol-5-yl}-2-methyl-acrylic acid;
5-{2-[5-(3,4-Dimethyl-phenyl)-thiophen-2-yl]-1H-benzoimidazol-5-ylmethylene}-2-thioxo-thiozolidin-4-one;
5-{2-[4-(3,4-Dimethyl-phenyl)-thiophen-2-yl]-1H-benzoimidazol-5-ylmethylene}-2-thioxo-thiozolidin-4-one;
5-{2-[5-(4-tert-Butyl-phenyl)-furan-2-yl]-1H-benzoimidazol-5-ylmethylene}-2-thioxo-thiozolidin-4-one;
5-[2-(4′-tert-Butyl-biphenyl-3yl)-1H-benzoimidazol-5-ylmethylene]-2-thioxo-thiozolidin-4-one; and
5-[2-(4′-tert-Butyl-2-hydroxy-biphenyl-3yl)-1H-benzoimidazol-5-ylmethylene]-2-thioxo-thiozolidinone;
and pharmaceutically acceptable salts, hydrates, solvates and esters thereof.

Compounds of Formula (I) are included in the pharmaceutical compositions of the invention and used in the methods of the invention.

By the term “protected hydroxy” or “protected —OH” as used herein, is meant the alcoholic or carboxylic-OH groups which can be protected by conventional blocking groups in the art such as described in “Protective Groups In Organic Synthesis” by Theodora W. Greene, Wiley-Interscience, 1981, New York. Compounds containing protected hydroxy groups may also be useful as intermediates in the preparation of the pharmaceutically active compounds of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4methylenedioxyphenyl, pyridine, biphenyl, quinoline, pyrimidine, quinazoline, thiophene, furan, pyrrole, pyrazole, imidazole and tetrazole.

When referring to compounds of Formula (I) and (II), the term “substituted” as used herein, unless otherwise defined, is meant that the subject chemical moiety has one or more substituents selected from the group consisting of: —CO₂R²⁰, aryl, —C(O)NHS(O)₂R²⁰, —NHS(O)₂R²⁰, hydroxyalkyl, alkoxy, —C(O)NR²¹R²², acyloxy, alkyl, amino, N-acylamino, hydroxy, —(CH₂)_{g}C(O)OR⁸, —S(O)ₙR⁸, nitro, tetrazole, cyano, oxo, halogen, trifluoromethyl, protected —OH, a heterocyclic methylene substituent as represented by Formula (III), 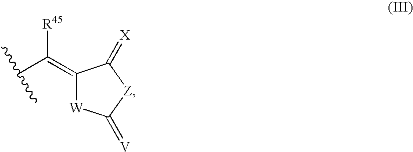
and a substituent as represented by Formula (VII), 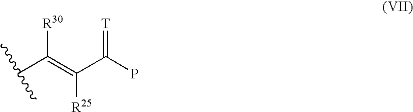
where g is 0-6; R⁸ is hydrogen or alkyl; R²⁰ is selected form hydrogen, C₁-C₄alkyl, aryl and trifluoromethyl; R²¹ and R²² are independently selected form hydrogen, C₁-C₄alkyl, aryl and trifluoromethyl; V, W, X and Z are each independently selected from O, S, and NR¹⁶, where R¹⁶ is selected from: hydrogen, alkyl, cycloalkyl, C₁-C₁₂aryl, substituted alkyl, substituted cycloalkyl and substituted C₁-C₁₂aryl; R¹⁰ is selected from: hydrogen, alkyl, cycloalkyl, C₁-C₁₂aryl, substituted alkyl, substituted cycloalkyl and substituted C₁-C₁₂aryl; R²⁵ and R³⁰ are independently selected from: hydrogen, alkyl, halogen, cycloalkyl, C₁-C₁₂aryl, substituted alkyl, substituted cycloalkyl and substituted C₁-C₁₂aryl; R⁴⁵ is selected from: hydrogen, alkyl, halogen, cycloalkyl, C₁-C₁₂aryl, substituted alkyl, substituted cycloalkyl and substituted C₁-C₁₂aryl; and n is 0-2.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, 4-hydroxy-cyclohexyl, 2-ethylcyclohexyl, propyl 4-methoxycyclohexyl, 4-methoxycyclohexyl, 4-carboxycyclohexyl, cyclopropyl and cyclopentyl.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifuloromethyl, acyloxy, amino, N-acylamino, hydroxy, —(CH₂)_{g}C(O)OR⁸, —S(O)ₙR⁸, nitro, cyano, halogen and protected —OH, where g is 0-6, R⁸ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein is meant a linear or branched, saturated or unsaturated hydrocarbon chain, and unless otherwise defined, the carbon chain will contain from 1 to 12 carbon atoms. Examples of alkyl substituents as used herein include: —CH₃, —CH₂—CH₃, —CH₂—CH₂—CH₃, —CH(CH₃)₂, —C(CH₃)₃, —(CH₂)₃—CH₃, —CH₂—CH(CH₃)₂, —CH(CH₃)—CH₂—CH₃, —CH═CH₂, and —C.C—CH₃.

By the term “treating” and derivatives thereof as used herein, is meant prophylatic and therapeutic therapy.

All publications, including but not limited to patents and patent applications, cited in this specification are herein incorporated by reference as though fully set forth.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The novel compounds of Formulas I and II are prepared by methods analogous to those shown in Schemes I to m below. All of the starting materials are commercially available or are readily made from commercially available starting materials by those of skill in the art.

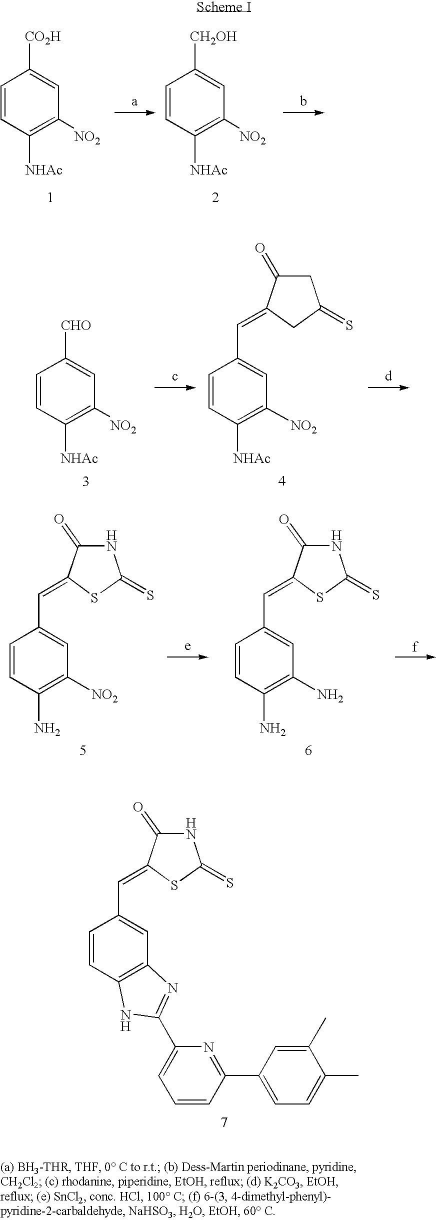

Compounds of Formula (I) and (II) can be prepared in a manner analogous to those shown in Scheme I. An appropriately functionalized benzoic acid, such as 1-Scheme I, is reduced with borane-THF complex or a powerful hydride donor reagent, such as lithium aluminum hydride or diisobutylaluminum hydride, in a suitable solvent, such as tetrahydrofuran. The resultant alcohol 2-Scheme I is then converted to the corresponding aldehyde 3-Scheme I by way of oxidation with Dess-Martin periodinane in dichloromethane or another suitable oxidant, such as pyridinium chlorochromate or chromium trioxide-pyridine complex. A subsequent Knoevenagel condensation of 3-Scheme I [Knovenagel, Chemische Berichte 29: 172 (1896)] with an active methylene compound, such as rhodanine, in the presence of a catalytic amount of a suitable amine base, such as piperidine, gives an olefin, such as 4-Scheme I. Removal of the N-acetyl protecting group is acomplished either under basic conditions using potassium carbonate in ethanol at reflux or acidic conditions, such as aqueous hydrochloric acid at reflux, to give the corresponding nitroaniline, such as 5-Scheme I. The nitro group is reduced using either a reducing metal, such as tin (II) chloride in hydrochoric acid, or catalytic hydrogenation with an appropriate catalyst, such as platinum oxide, to afford the desired dianiline 6-Scheme I. Conversion of the latter to a compound of Formula (I) or (II), such as 7-Scheme I, is achieved via either cyclo-condensation with a suitable biaryl aldehyde, such as 6-(3,4-dimethyl-phenyl)-pyridine-2-carbaldehyde, in the presence of a suitable aromatizing agent, such as aqueous sodium hydrogensulfite or 1,4-benzoquinone, in ethanol, or condensation with an appropriate biaryl carboxylic acid with heating in either formic, hydrochloric or polyphosphoric acid.

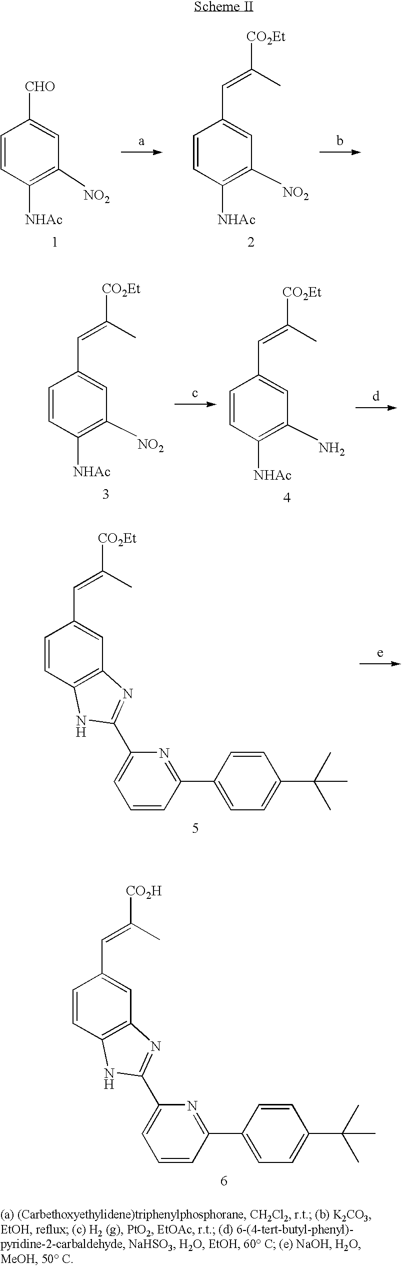

Alternatively, the compounds for Formula (I) can be prepared as shown in Scheme II. Olefination of an appropriate aldehyde, such as 1-Scheme II, using a Horner-Wadsworth-Emmons reaction, or preferably, a salt free Wittig reaction with an appropriate phophorane, such as (carbethoxyethylidene)triphenylphosphorane, gives the corresponding olefin, 2-Scheme II. Removal of the N-acetyl protecting group is accomplished either under basic conditions using potassium carbonate in ethanol at reflux or acidic conditions, such as aqueous hydrochloric acid at reflux, to give the corresponding nitroaniline, such as 3-Scheme II. The nitro group is then reduced using catalytic hydrogenation with an appropriate catalyst, such as platinum oxide, yielding dianiline, such as 4-Scheme II. Conversion of the latter to a compound of Formula (I) or (II), represented by 5-Scheme II, is achieved via cyclo-condensation with a suitable biaryl aldehyde, such as 6-(4-tert-butyl-phenyl)-pyridine-2-carbaldehyde, in the presence of a suitable aromatizing agent, such as aqueous sodium hydrogensulfite or 1,4benzoquinone, with heating in ethanol. Saponification of the ester moiety in 5-Scheme II under acidic or, preferably, basic conditions using an aqueous base, such as sodium hydroxide in aqueous methanol, then gives rise to a compound of Formula (I) or (II), such as 6-Scheme II.

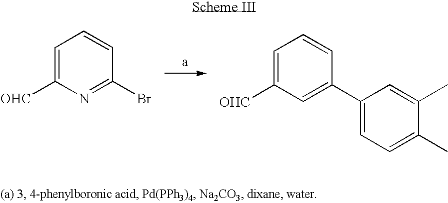

Scheme IV outlines the formation biaryl aldehydes used in Schemes I-II. An appropriately functionalized aryl halide is reacted with a suitable arylboronic acid, such as 3,4-dimethylboronic acid, in the presence of a catalyst, preferably tetrakistriphenylphosphino palladium, and a base, such as sodium carbonate or triethylamine, in a suitable solvent, such as aqueous 1,4-dioxane or dimethylformamide, to give a substituted aryl compound, such as 2-Scheme III.

In preparing the presently invented compounds of Formula (I), the following novel intermediates are prepared:
N-[2-Nitro-4-(4-oxo-2-thioxo-thiazolidin-5-ylidenemethyl)-phenyl]-acetamide;
5-(4-Amino-3-nitro-benzylidene)-2-thioxo-thiazolidin-4-one;
5-(3,4-Diamino-benzylidene)-2-thioxo-thiazolidin-4-one;
(E)-3-(4-Acetylamino-3-nitro-phenyl)-2-methyl-acrylic acid ethyl ester;
(E)-3-(4-Amino-3-nitro-phenyl)-2-methyl-acrylic acid ethyl ester;
(E)-3-(3,4-Diamino-phenyl)-2-methyl-acrylic acid ethyl ester;
6(3,4-Dimethyl-phenyl)-pyridine-2-carbaldehyde;
6-(3,4-Dichloro-phenyl)-pyridine-2-carbaldehyde;
6-(4-tert-Butyl-phenyl)-pyridine-2-carbaldehyde;
4′-tert-Butyl-2-methoxy-biphenyl-3-carbaldehyde; and
4′-tert-Butyl-2-hydroxy-biphenyl-3-carbaldehyde.

The treatment of thrombocytopenia, as described herein, is accomplished by increasing the production of platelets.

By the term “co-administering” and derivatives thereof as used herein is meant either simultaneous administration or any manner of separate sequential administration of a TPO mimetic compound, as described herein, and a further active ingredient or ingredients, known to treat thrombocytopenia, including chemotherapy-induced thrombocytopenia and bone marrow transplantation and other conditions with depressed platelet production. The term further active ingredient or ingredients, as used herein, includes any compound or therapeutic agent known to or that demonstrates advantageous properties when administered with TPO or a TPO mimetic. Preferably, if the administration is not simultaneous, the compounds are administered in a close time proximity to each other. Furthermore, it does not matter if the compounds are administered in the same dosage form, e.g. one compound may be administered topically and another compound may be administered orally.

Examples of a further active ingredient or ingredients for use in combination with the presently invented TPO mimetic compounds include but are not limited to: chemoprotective or myeloprotective agents such as G-CSF, BB 10010 (Clemons et al., Breast Cancer Res. Treatment, 1999, 57, 127), amifostine (Ethyol) (Fetscher et al., Current Opinion in Hemat., 2000, 7, 255-60), SCF, IL-11, MCP-4, IL-1-beta, AcSDKP (Gaudron et al., Stem Cells, 1999, 17, 100-6), TNF-a, TGF-b, MIP-1a (Egger et al., Bone Marrow Transpl., 1998, 22 (Suppl. 2), 34-35), and other molecules identified as having anti-apoptotic, survival or proliferative properties.

Tpo has been demonstrated to act as a mobilizer of stem cells into the peripheral blood (Neumann T. A. et al., Cytokines, Cell. & Mol. Ther., 2000, 6, 47-56). This activity can synergize with stem cell mobilizers such as G-CSF (Somolo et al., Blood, 1999, 93, 2798-2806). The TPO mimetic compounds of the present invention are thus useful in increasing the numbers of stem cells in circulation in donors prior to leukapheresis for hematopoietic stem-cell transplantation in patients receiving myelo-ablative chemotherapy.

Likewise, TPO stimulates growth of myeloid cells, particularly those of granulocytelmacrophage lineage (Holly et al., U.S. Pat. No. 5,989,537). Granulocyte/macrophage progenitors are cells of the myeloid lineage that mature as neutrophils, monocytes, basophils and eosinophils. The compounds described in the present invention have thus therapeutic utility in stimulating the poliferation of neutrophils in patients with neutropenic conditions.

Additional examples of a further active ingredient or ingredients for use in combination with the presently invented TPO mimetic compounds include but are not limited to: stem cell, megakaryocyte, neutrophil mobilizers such as chemotherapeutic agents (i.e., cytoxan, etoposide, cisplatin, Ballestrero A. et al., Oncology, 2000, 59, 7-13), chemokines, IL-8, Gro-beta (King, A. G. et al. J. Immun., 2000, 164, 3774-82), receptor agonist or antagonist antibodies, small molecule cytokine or receptor agonists or antagonists, SCF, Flt3 ligand, adhesion molecule inhibitors or antibodies such as: anti-VLA-4 (Kikuta T. et al., Exp. Hemat., 2000, 28, 311-7) or anti-CD44 (Vermeulen M. et al., Blood, 1998, 92, 894-900), cytokine/chemokine/interleukin or receptor agonist or antagonist antibodies, MCP-4 (Berkhout T A., et al., J. Biol. Chem., 1997, 272, 16404-16413; Uguccioni M. et al., J. Exp. Med., 1996, 183, 2379-2384).

Because the pharmaceutically active compounds of the present invention are active as TPO mimetics they exhibit therapeutic utility in treating thrombocytopenia and other conditions with depressed platelet production.

By the term “thrombocytopenia” and derivatives thereof as used herein is to be broadly interpreted as any decrease in the number of blood platelets below what is considered normal or desired for a healthy individual. Thrombocytopenia is known to have many causative factors, including but not limited to, radiation therapy, chemotherapy, immune therapy, immune thrombocytopenic purpura (ITP, Bussel J. B., Seminars in Hematology, 2000, 37, Suppl 1, 1-49), myelodysplastic syndrom (MDS), aplastic anemia, AML, CML, viral infections (including, but not limited to; HIV, hepatitis C, parvovirus) liver disease, myeloablation, bone marrow transplant, stem cell transplant, peripheral blood stem cell transplant, progenitor cell defect, polymorphisms in stem cells and progenitor cells, defects in Tpo, neutropenia (Sawai, N. J. Leukocyte Biol., 2000, 68, 137-43), dendritic cell mobilization (Kuter D. J. Seminars in Hematology, 2000, 37, Suppl 4, 41-49), proliferation, activation or differentiation. The pharmaceutically active compounds of this invention are useful in treating thrombocytopenia regardless of the factor or factors causing the condition. The pharmaceutically active compounds of this invention are also useful in treating thrombocytopenia when the causative factor or factors of the condition are unknown or have yet to be identified.

Prophylactic use of the compounds of this invention is contemplated whenever a decrease in blood or blood platelets is anticipated. Prophylactic use of the compounds of this invention results in a build up of platelets or a commencement of platelet production prior to an anticipated loss of blood or blood platelets. Prophylactic uses of the compounds of this invention includes but is not limited to transplant surgery, surgery, anesthesia prior to child birth and gut protection.

Human dendritic cells have been shown to express the TPO receptor (Kumamoto et al., Br. J. Haem, 1999, 105, 1025-1033) and TPO is a potent mobilizer of dendritic cells. The TPO mimetic compounds of the current invention are also useful as a vaccine adjuvant in that they increase the activity and mobility of dendritic cells. The pharmaceutically active compounds of this invention are useful as an immunological adjuvant, given in combination with an orally, transdermally or subcutaneously delivered vaccine and/or immunomodulator, by increasing the activity and mobility of dendritic cells.

Tpo is known to have various effects including anti-apototic/survival effects on megakaryocytes, platelets and stem cells, and proliferative effects on stem cells and megakaryocytic cells (Kuter D. J. Seminars in Hematology, 2000, 37, 41-9). These Tpo activities effectively increase the number of stem and progenitor cells so that there is synergistic effects when Tpo is used in conjunction with other cytokines that induce differentiation.

The TPO mimetic compounds of the current invention are also useful in acting on cells for survival or proliferation in conjunction with other agents known to act on cells for survival or proliferation. Such other agents include but are not limited to: G-CSF, GM-CSF, TPO, M-CSF, EPO, Gro-beta, IL-11, SCF, FLT3 ligand, LIF, IL-3, IL-6, IL-1, Progenipoietin, NESP, SD-01, or IL-5 or a biologically active derivative of any of the aforementioned agents, KT6352 (Shiotsu Y. et al., Exp. Hemat. 1998, 26, 1195-1201), uteroferrin (Laurenz J C., et al. Comp. Biochem. & Phys., Part A. Physiology., 1997, 116, 369-77), FK23 (Hasegawa T., et al. Int. J. Immunopharm., 1996, 18 103-112) and other molecules identified as having anti-apoptotic, survival or proliferative properties for stem cells, progenitor cells, or other cells expressing Tpo Receptors.

In determining potency as TPO mimetics, the following assays are employed:

Proliferation Assays

Compounds of the present invention were tested for potency as mimetics of TPO in an in vitro proliferation assay using the murine BaF3 cell line transfected with human Tpo-R. Survival and growth is dependent on the presence of TPO.

Compounds of this invention were tested for potency as mimetics of TPO in an in vitro proliferation assay using the human UT7TPO cell line. UT7TPO cells are a human megakaryoblastic cell line that express Tpo-R, whose survival and growth is dependent on the presence of TPO (Komatsu et al. Blood 1996, 87,4552).

Differentiation Assay

Likewise, compounds of this invention are also tested for activity as mimetics of TPO in a human bone marrow cell megakaryocyte maturation assay. In this assay, purified human CD34+ progenitor cells are incubated in liquid culture with test compounds for 10 days and the number of cells expressing the transmembrane glycoprotein CD41 (gpIIb), a megakaryocytic marker, are then measured by flow cytometry (see Cwirla, S. E. et al Science, 1997, 276, 1696).

Some of the preferred compounds within the scope of the invention showed activation from about 20% to 110% of control at a concentration of 1.5 to 6 uM in the proliferation of BaF3 cells in the above assay.

Within the scope of the invention 5-{2-[6-(3,4Dichloro-phenyl)-pyridin-2-yl]-1H-benzoimidazol-5-ylmethylene}-2-thioxo-thiozolidin-4-one showed an activation of about 20% of control (control is the maximal response to TPO) at a concentration of 2 uM in the proliferation of BaF3 cells in the above assay.

The pharmaceutically active compounds within the scope of this invention are useful as TPO mimetics in mammals, particularly humans, in need thereof.

The present invention therefore provides a method of treating thrombocytopenia and other conditions with depressed platelet production, which comprises administering a compound of Formula (I) or a pharmaceutically acceptable salt, hydrate, solvate or ester thereof in a quantity effective to enhance platelet production. The compounds of Formula (I) also provide for a method of treating the above indicated disease states because of their demonstrated ability to act as TPO mimetics. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid;. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a, pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a TPO mimetic, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular TPO mimetic in use, the strength of the preparation, the mode of administration, and the advancement of the disease condition. Additional factors depending on the particular patient being treated will result in a need to adjust dosages, including patient age, weight, diet, and time of administration.

The method of this invention of inducing TPO mimetic activity in mammals, including humans, comprises administering to a subject in need of such activity an effective TPO mimetic amount of a pharmaceutically active compound of the present invention.

The invention also provides for the use of a compound of Formula (I) in the manufacture of a medicament for use as a TPO mimetic.

The invention also provides for the use of a compound of Formula (I) in the manufacture of a medicament for use in therapy.

The invention also provides for the use of a compound of Formula (I) in the manufacture of a-medicament for use in enhancing platelet production.

The invention also provides for the use of a compound of Formula (I) in the manufacture of a medicament for use in treating thrombocytopenia.

The invention also provides for a pharmaceutical composition for use as a TPO mimetic which comprises a compound of Formula (I) and a pharmaceutically acceptable carrier.

The invention also provides for a pharmaceutical composition for use in the treatment of thrombocytopenia which comprises a compound of Formula (I) and a pharmaceutically acceptable carrier.

The invention also provides for a pharmaceutical composition for use in enhancing platelet production which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat thrombocytopenia, including chemotherapy-induced thrombocytopenia and bone marrow transplantation and other conditions with depressed platelet production, or compounds known to have utility when used in combination with a TPO mimetic.

Contemplated Equivalents—It will be appreciated by the person of ordinary skill in the art that the compounds of Formulas I and II may also exist in tautomeric forms. For example, in Formula II, the double bond that is drawn between the nitrogen atom and the 2 position of the imidazole ring exists between the 2 position of the imidazole ring and the alternative nitrogen atom of the ring. Tautomeric forms of the compounds of Formulas I and II are exemplified by the following Formula (VI): 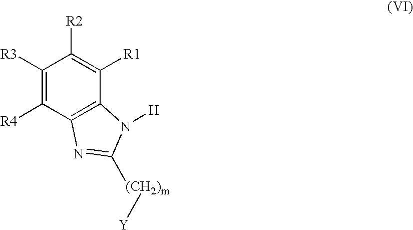
where the ‘R’, m and Y groups are as defined above. All such compounds are included in the scope of the invention and inherently included in the definition of the compounds of Formulas I and II.

Without further elaboration, it is believed that one skilled in the art can, using the preceding description, utilize the present invention to its fullest extent. The following Examples are, therefore, to be construed as merely illustrative and not a limitation of the scope of the present invention in any way.

### Experimental Details

### EXAMPLE 1

### Preparation of 5-{2-[6-(3,4-dichloro-phenyl)-pyridin-2-yl]-1H-benzoimidazol-5-ylmethylene}-2-thioxo-thiozolidin-4-one

### a) N-[2-Nitro-4-(4-oxo-2-thioxo-thiazolidin-5-ylidenemethyl)-phenyl]-acetamide

To a stirred suspension of N-(4-formyl-2-nitro-phenyl)-acetamide (prepared according to the procedure of Tanaka et al. Chem. Pharm. Bull. 1994, 42, 560; 200 mg, 0.961 mmol) and rhodanine (141 mg, 1.06 mmol) in absolute ethanol (10 mL) at ambient temperature was added piperidine (10 μL, 0.096 mmol). The reaction mixture was heated to reflux and stirred overnight, at which time it was cooled to ambient temperature and filtered. The filter cake was washed with diethyl ether and dried under vacuum at 50° C., yielding 293 mg (94% yield) of an orange solid. ¹H NMR [400 MHz, (CH₃)₂SO] δ 10.40 (s, 1 H), 8.24 (br s, 1 H), 8.03 (d, J=2.0 Hz, 1 H), 7.78 (dd, J=8.6, 2.0 Hz, 1 H), 7.70 (d, J=8.6 Hz, 1 H), 7.18 (s, 1 H), 2.08 (s, 3 H).

### b) 5-(4-Amino-3-nitro-benzylidene)-2-thioxo-thiazolidin-4-one

To a stirred suspension of the compound of Example 1(a) (190 mg, 0.588 mmol) in absolute ethanol (11 mL) at ambient temperature was added potassium carbonate (408 mg, 2.95 mmol). The reaction mixture was stirred at reflux for 2.5 h, at which time it was cooled and filtered. The solvent was removed under reduced pressure, and the resultant dark red residue was suspended in water (10 mL) and filtered. The filter cake was washed with water (5 mL) and dried under vacuum at 50° C., yielding 152 mg (92%) of a brick-colored solid. ¹H NMR [400 MHz, (CH₃)₂SO] δ 8.09 (d, J=1.8 Hz, 1 H), 7.75 (br s, 2 H), 7.56 (br dd, J=8.6, 2.0 Hz, 1 H), 7.06 (d, J=8.8 Hz, 1 H), 7.04 (s, 1 H).

### c) 5-(3,4Diamino-benzylidene)-2-thioxo-thiazolidin-4-one

To a stirred suspension of the compound of Example 1(b) (38.3 mg, 0.136 mmol) in concentrated hydrochloric acid (3 mL) was added tin (II) chloride (77.4 mg, 0.408 mmol). The reaction mixture was then stirred at 100° C. for a duration of 2.5 h, at which time it was cooled to ambient temperature and filtered. The filter cake was washed with cold water (3 mL) and dried under vacuum at 50° C., yielding 33 mg of a pale orange solid. ¹H NMR [400 MHz, (CH₃)₂SO] δ 7.48 (s, 1 H), 7.39-7.34 (m, 2 H), 6.92 (d, J=8.10 Hz, 1 H).

### d) 6-(3,4-Dichloro-phenyl)-pyridine-2-carbaldehyde

To a stirred solution of 6-bromo-pyridine-2-carbaldehyde (prepared according to the procedure of Cai et al. Tetrahedron Lett. 1996, 37, 2537; 200 mg, 1.08 mmol) in 1,4-dioxane (8 mL) at ambient temperature was added tetrakistriphenylphosphino palladium (16.2 mg, 13.8 μmol). After complete dissolution of the latter (3 min), 3,4-dichlorophenylboronic acid (191.4 mg, 1.08 mmol) was added in one portion, followed by aqueous sodium carbonate (2 M, 1.08 mL, 2.16 mmol), and the resultant mixture was heated at reflux overnight. After cooling to ambient temperature, the solvent was removed in vacuo. The resultant residue was purified by gradient flash chromatography on silica gel (1:1 dichloromethan/hexanes→4:1 dichloromethane/hexanes) to afford the product (228 mg, 84% yield) as a white crystalline solid. ¹H NMR (400 MHz, CDCl₃) δ 10.16 (s, 1 H), 8.24 (d, J=2.1 Hz, 1 H), 8.00-7.92 (complex m, 4 H), 7.59 (d, J=8.3 Hz, 1 H).

### e) 5-{2-[6-(3,4-Dichloro-phenyl)-pyridin-2-yl]-1H-benzoimidazol-5-ylmethylene}-2-thioxo-thiozolidin-4-one

To a stirred suspension of the compound of Example 1(c) (16 mg 63.7 μmol) in absolute ethanol (2 mL) at 60° C. was added water (0.3 mL), followed by sodium hydrogensulfite (30 mg). The deep red solution was stirred for 5 min, at which time a solution of the compound of Example 1(d) in absolute ethanol (2.5 mL) was added dropwise over 15 min. The resultant mixture was stirred at 60° C. for 3 h, cooled to ambient temperature, and then filtered. The filter cake was washed with water (2 mL) then diethyl ether (5 mL) and dried under vacuum at 50° C. to afford the product (26.8 mg, 87% yield) as a pale orange solid. MS (ES+) m/z 483.2 (M+H)⁺.

### EXAMPLE 2

### Preparation of 5-{2-[6-(3,4-dimethyl-phenyl)-pyridin-2-yl]-1H-benzoimidazol-5-ylmethylene}-2-thioxo-thiozolidin-4-one

### a) 6-(3,4-Dimethyl-phenyl)-pyridine-2-carbaldehyde

The title compound was prepared in a manner analogous to the preparation of the compound of Example 1(d), except substituting 3,4-dichlorophenylboronic acid for 3,4-dimethylphenylboronic acid. ¹H NMR (400 MHz, CDCl₃) δ 10.17 (s, 1 H), 7.94-7.85 (complex m, 4 H), 7.80 (dd, J=7.8, 1.5 Hz, 1 H), 7.27 (d, J=7.8 Hz, 1 H), 2.37 (s, 3 H), 2.34 (s, 3 H).

### b) 5-{2-[6-(3,4-Dimethyl-phenyl)-pyridin-2-yl]-1H-benzoimidazol-5-ylmethylene}-2-thioxo-thiozolidin-4-one

The title compound was prepared in a manner analogous to the preparation of the compound of Example 1(e), except substituting the compound of Example 1(d) for the compound of Example 2(a). MS (ES+) m/z 443.2 (M+H)⁺.

### EXAMPLE 3

### Preparation of (E)-3-{2-[6-(4-tert-butyl-phenyl)-pyridin-2-yl]-1H-benzoimidazol-5-yl}-2-methyl-acrylic acid

### a) (E)-3-(4-Acetylamino-3-nitro-phenyl)-2-methyl-acrylic acid ethyl ester

To a solution of N-(4formyl-2-nitro-phenyl)-acetamide (406 mg, 1.95 mmol) in dichloromethane (17 mL) at ambient temperature was added a solution of (carbethoxyethylidene)triphenylphosphorane (919 mg, 2.54 mmol) in dichloromethane (17 mL) dropwise over 15 min. The reaction mixture was stirred at ambient temperature overnight and then concentrated in vacuo. The resultant residue was purifed by flash chromatography on silica gel (40:1 dichloromethane/methanol) to give the title compound (560 mg, 98% yield) as a lemon-yellow solid. ¹H NMR (400 MHz, CDCl₃) δ 10.37 (s, 1 H), 8.82 (d, J=9.1 Hz, 1 H), 8.26 (d, J=1.8 Hz, 1 H), 7.68 (dd, J=8.8, 2.0 Hz, 1 H), 7.60 (s, 1 H), 4.28 (q, 7.1 Hz, 2 H), 2.31 (s, 3 H), 2.13 (d, J=1.3 Hz, 3 H), 1.35 (t, J=7.1 Hz, 3 H).

### b) (E)-3-(4-Amino-3-nitro-phenyl)-2-methyl-acrylic acid ethyl ester

To a solution of the compound of Example 3(a) (543 mg, 1.86 mmol) in absolute ethanol (20 mL) was added potassium carbonate (514 mg, 3.72 mmol). The resultant mixture was stirred at reflux for I h, at which time it was cooled to ambient temperature, filtered, and concentrated in vacuo. The residue was taken up in water (50 mL), and the resultant solution acidified with concentrated hydrochloric acid to pH 4 and extracted with dichloromethane (3×50 mL). The combined organic extracts were washed with brine (50 mL), dried over MgSO₄, and concetrated in vacuo to provide the title compound (421 mg, 91% yield) as an orange solid. ¹H NMR (400 MHz, CDCl₃) δ 8.23 (d, J=1.8 Hz, 1 H), 7.54 (s, 1 H), 7.45 (dd, J=8.6, 2.0 Hz, 1 H), 6.84 (d, J=8.6 Hz, 1 H), 6.25 (br s, 2 H), 4.26 (q, 7.3 Hz, 2 H), 2.14 (d, J=1.5 Hz, 3 H), 1.34 (t, J=7.1 Hz, 3 H).

### c) (E)-3-(3,4-Diamino-phenyl)-2-methyl-acrylic acid ethyl ester

A solution of the compound of Example 3(b) (121.6 mg, 4.86 mmol) in ethyl acetate (10 mL) was hydrogenated at ambient temperature and pressure with catalytic platinum (IV) oxide. The course of the reaction was monitored by TLC every 10 min; complete consumption of the starting material was achieved in 40 min. The hydrogen was vented, and the catalyst removed by filtration. The filtrate was concetrated in vacuo to afford the title compound (108 mg, quantitative yield) as a pink oil which was submitted to the next step without further purification. ¹H NMR (400 MHz, CDCl₃) δ 7.56 (s, 1 H), 6.86 (dd, J=8.1, 1.8 Hz, 1 H), 6.83 (d, J=1.8 Hz, 1 H), 6.70 (d, J=8.1 Hz, 1 H), 4.24 (q, J=7.1 Hz, 1 H), 3.57 (br s, 2 H), 3.38 (br s, 2 H), 2.13 (d, J=1.2 Hz, 3 H), 1.33 (t, J=7.1 Hz, 3 H).

### d) 6-(4-tert-Butyl-phenyl)-pyridine-2-carbaldehyde

The title compound was prepared in a manner analogous to the preparation of the compound of Example 1(d), except substituting 3,4-dichlorophenylboronic acid for 4-tert-butylphenylboronic acid. ¹H NMR (400 MHz, CDCl₃) δ 10.16 (s, 1 H), 8.02 (app d, J=8.6 Hz, 2 H), 7.96-7.86 (series of m, 3 H), 7.55 (app d, J=8.6 Hz, 2 H), 1.38 (s, 9 H).

### e) (E)-3-}2-[6-(4-tert-Butyl-phenyl)-pyridin-2-yl]-1H-benzoimidazol-5-yl}-2-methyl-acrylic acid ethyl ester

The title compound was prepared in a manner analogous to the preparation of the compound of Example 1(e), except substituting the compound of Example 1(c) for the compound of Example 3(c) and substituting the compound of Example 1(d) for the compound of Example 3(d). ¹H NMR (400 MHz, CDCl₃) δ 10.76 (br s, 1 H), 8.34 (dd, J=7.8, 0.8 Hz, 1 H), 8.02 (d, J=2.3 Hz, 1 H), 8.00 (d, J=2.3 Hz, 1 H), 7.96-7.82 (series of m, 3 H), 7.78 (dd, J=8.1, 0.8 Hz, 1 H), 7.61-7.50 (m, 3 H), 7.36 (d, J=8.3 Hz, 1 H), 4.30 (q, J=7.1 Hz, 2 H), 2.21 (dd, J=4.3, 1.3 Hz, 3 H), 1.39-1.37 (m, 12 H).

### f) (E)-3-{2-[6-(4-tert-butyl-phenyl)-pyridin-2-yl]-1H-benzoimidazol-5-yl}-2-methyl-acrylic acid

To a solution of the compound of Example 3(e) (30 mg, 68.2 μmol) in methanol (2 mL) was added aqueous potassium hydroxide (2 M, 0.34 mL, 0.680 mmol), and the resultant mixture was stirred at 50° C. for 24 h. The reaction mixture then was neutralized with 6 N aqueous hydrochloric acid, concentrated in vacuo, and purified by gradient flash chromatography on silica gel (30:1→10:1 dichloromethane/methanol) to give the product (25 mg, 89% yield) as a glassy solid. ¹H NMR (400 MHz, CDCl₃) δ 8.23 (app d, J=8.4 Hz, 2 H), 8.19 (dd, J=6.8, 2.0 Hz, 1 H), 8.01-7.94 (m, 2 H), 7.86 (s, 1 H), 7.79 (s, 1 H), 7.71 (d, J=8.6 Hz, 1 H), 7.57 (app d, J=8.6 Hz, 2 H), 7.40 (dd, J=8.4, 1.2 Hz, 1 H), 2.20 (d, J=1.6 Hz, 3 H), 1.39 (s, 9 H).

### EXAMPLE 4

### Preparation of 5-{2-[5-(3,4-Dimethyl-phenyl)-thiophen-2-yl}-1H-benzoimidazol-5-ylmethylene}-2-thioxo-thiozolidin-4-one

The title compound was prepared in a manner analogous to the preparation of the compound of Example 1(e), except substituting the compound of Example 1(d) for 5-(3,4-dimethyl-phenyl)-thiophene-2-carbaldehyde (prepared according to WO139773). ¹H NMR [400 MHz, (CD₃)₂SO] δ 13.28 (br s, 1 H), 7.86 (br s, 1 H), 7.77-7.61 (m, 3 H), 7.58 (d, J=3.8 Hz, 1 H), 7.54 (br s, 1 H), 7.50-7.39 (m, 2 H), 2.29 (s, 3 H), 2.26 (s, 3 H).

### EXAMPLE 5

### Preparation of 5-{2-[4-(3,4-Dimethyl-phenyl)-thiophen-2-yl]-1H-benzoimidazol-5-ylmethylene}-2-thioxo-thiozolidin-4-one

The title compound was prepared in a manner analogous to the preparation of the compound of Example 1(e), except substituting the compound of Example 1(d) for 4-(3,4-dimethyl-phenyl)-thiophene-2-carbaldehyde (prepared according to WO139773). ¹H NMR [400 MHz, (CD₃)₂SO] δ 13.31 (br s, 1 H), 8.30 (br s, 1 H), 8.03 (s, 1 H), 7.90-7.63 (m, 3 H), 7.53 (br s, 1 H), 7.51-7.38 (m, 2 H), 7.24 (d, J=7.8 Hz, 1 H), 2.30 (s, 3 H), 2.26 (s, 3 H).

### EXAMPLE 6

### Preparation of 5-{2-[5-(4-tert-Butyl-phenyl)-furan-2-yl]-1H-benzoimidazol-5-ylmethylene}-2-thioxo-thiozolidin-4one

The title compound was prepared in a manner analogous to the preparation of the compound of Example 1(e), except substituting the compound of Example 1(d) for 5-(4-tert-butyl-phenyl)-furan-2-carbaldehyde (prepared according to WO139773). ¹H NMR [400 MHz, (CD₃)₂SO] δ 7.89 (complex m, 4 H), 7.75 (d, J=8.3 Hz, 1 H), 7.53 (app d, J=8.8 Hz, 2 H), 7.49 (dd, J=8.6. 1.5 Hz, 1 H), 7.39 (d, J=3.8 Hz, 1 H), 7.18 (d, J=3.8 Hz, 1 H), 1.33 (s, 9 H).

### EXAMPLE 7

### Preparation of 5-[2-(4′-tert-Butyl-biphenyl-3yl)-1H-benzoimidazol-5-ylmethylene]-2-thioxo-thiozolidin-4-one

The title compound was prepared in a manner analogous to the preparation of the compound of Example 1(e), except substituting the compound of Example 1(d) for 4′-tert-butyl-biphenyl-3-carbaldehyde (prepared according to WO139773). ¹H NMR [400 MHz, (CD₃)₂SO] δ 13.34 (br s, 1 H), 8.49 (s, 1 H), 8.19 (d, J=8.1 Hz, 1 H), 7.82 (br d, J=8.1 Hz, 2 H), 7.77-7.70 (complex m, 4 H), 7.75 (t, J=7.8 Hz, 1 H), 7.55 (app d, J=8.3 Hz, 2 H), 7.47 (br d, J=7.6 Hz, 1 H), 1.35 (s, 9 H).

### EXAMPLE 8

### Preparation of 5-[2-(4′-tert-butyl-2-hydroxy-biphenyl-3yl)-1H-benzoimidazol-5-ylmethylene]-2-thioxo-thiozolidin-4one

### a) 4′-tert-Butyl-2-methoxy-biphenyl-3-carbaldehyde

The title compound was prepared in a manner analogous to the preparation of the compound of Example 1(d), except substituting 6-bromo-pyridine-2-carbaldehyde for 3-bromo-2-methoxy-benzaldehyde (prepared according to the procedure of Aristoff et al. Tetrahedron Lett. 1984, 25, 3955) and substituting 3,4-dichlorophenylboronic acid for 4-tert-butylphenylboronic acid. ¹H NMR (400 MHz, CDCl₃) δ 10.49 (d, J=0.8 Hz, 1 H), 7.83 (dd, J=7.8, 1.8 Hz, 1 H), 7.60 (dd, J=7.6, 1.8 Hz, 1 H), 7.54-7.50 (m, 2 H), 7.49-7.45 (m, 2 H), 7.27 (dt, J=7.6, 0.8 Hz, 1 H), 3.53 (s, 3 H), 1.38 (s, 9 H).

### b) 4′-tert-Butyl-2-hydroxy-biphenyl-3-carbaldehyde

To a solution of the compound in Example 8(a) [100 mg, 0.373 mmol) in dichloromethane (2 mL) at 0° C. was added a solution of boron trichloride (1 M, 1 mL, 1.00 mmol), and the resultant mixture was stirred at that temperature for 3.5 h. The reaction was quenched with water (2 mL) and extracted with diethyl ether (3×5 mL). The combined organic extracts were dried over MgSO₄ and concentrated in vacuo. Flash chromatography on silica gel (1:1 dichloromethane/hexanes) afforded the title compound (93 mg, 98% yield) as a colorless solid. ¹H NMR (400 MHz, CDCl₃) δ 11.55 (s, 1 H), 9.95 (s, 1 H), 7.63 (dd, J=7.6, 1.8 Hz, 1 H), 7.58-7.52 (m, 3 H), 7.51-7.46 (m, 2 H), 7.10 (t, J=7.6 Hz, 1 H), 1.37 (s, 9 H).

### c) 5-[2-(4′-tert-Butyl-2-hydroxy-biphenyl-3yl)-1H-benzoimidazol-5-ylmethylene]-2-thioxo-thiozolidin-4-one

The title compound was prepared in a manner analogous to the preparation of the compound of Example 1(e), except substituting the compound of Example 1(d) for the compound of Example 8(b). ¹H NMR [400 MHz, (CD₃)₂SO] δ 13.66 (br s, 1 H), 13.59 (br s, 1 H), 8.16-7.94 (m, 2 H), 7.94-7.73 (m, 2 H), 7.67-7.46 (complex m, 6 H), 7.13 (t, J=7.7 Hz, 1 H), 1.34 (s, 9 H).

### EXAMPLE 9

### Capsule Composition

An oral dosage form for administering a presently invented agonist of the TPO receptor is produced by filing a standard two piece hard gelatin capsule with the ingredients in the proportions shown in Table I, below.

TABLE I

| INGREDIENTS | AMOUNTS | |
|---|---|---|
| 5-{2-[6-(3,4-Dichloro-phenyl)-pyridin-2-yl]-1H- | 25 | mg |
| benzoimidazol-5-ylmethylene}-2-thioxo-thiozolidin- | | |
| 4-one; (Compound of Example 1) | | |
| Lactose | 55 | mg |
| Talc | 16 | mg |
| Magnesium Stearate | 4 | mg |

### EXAMPLE 10

### Injectable Parenteral Composition

An injectable form for administering a presently invented agonist of the TPO receptor is produced by stirring 1.5% by weight of 5-{2-[6(3,4-Dimethyl-phenyl)-pyridin-2-yl]-1H-benzoimidazol-5-ylmethylene}-2-thioxo-thiozolidin-4-one; (Compound of Example 2) in 10% by volume propylene glycol in water.

### EXAMPLE 11

### Tablet Composition

The sucrose, calcium sulfate dihydrate and a presently invented agonist of the TPO receptor, as shown in Table II below, are mixed and granulated in the proportions shown with a 10% gelatin solution. The wet granules are screened, dried, mixed with the starch, talc and stearic acid;, screened and compressed into a tablet.

TABLE II

| INGREDIENTS | AMOUNTS | |
|---|---|---|
| (E)-3-{2-[6-(4-tert-butyl-phenyl)-pyridin-2-yl]-1H- | 20 | mg |
| benzoimidazol-5-yl}-2-methyl-acrylic acid; | | |
| (Compound of Example 3) | | |
| calcium sulfate dihydrate | 30 | mg |
| sucrose | 4 | mg |
| starch | 2 | mg |
| talc | 1 | mg |
| stearic acid | 0.5 | mg |

While the preferred embodiments of the invention are illustrated by the above, it is to be understood that the invention is not limited to the precise instructions herein disclosed and that the right to all modifications coming within the scope of the following claims is reserved.

1. A compound represented by the following Formula (I): 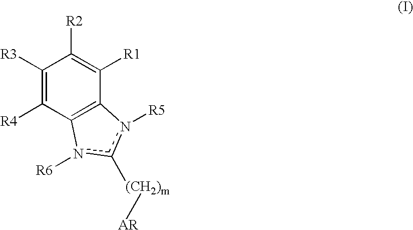
wherein:
the B ring has one double bond where indicated by the broken lines, provided that R⁵ is absent when the nitrogen attached thereto has a double bond and provided that R⁶ is absent when the nitrogen attached thereto has a double bond;
R¹, R², R³ and R⁴ are each independently selected from hydrogen,
—(CH₂)ₚOR¹⁰, —C(O)OR¹⁰, formyl, nitro, cyano, halogen, aryl, substituted aryl, substituted alkyl, —S(O)ₙR¹⁰, cycloalkyl, —NR¹¹R¹², protected —OH,
—CONR¹¹R¹², phosphonic acid, sulfonic acid, phosphinic acid, —SO₂NR¹¹R¹², a heterocyclic methylene substituent as represented by Formula (III), 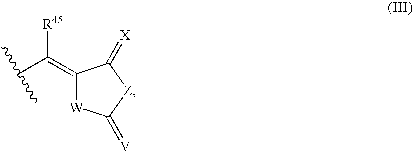
and
a substituent as represented by Formula (VII), 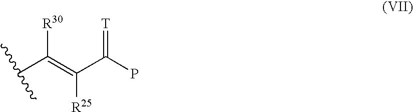
where,
p is 0-6,
n is 0-2,
W and Z are each independently selected from C, O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
V and X are each independently selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R¹⁰ is selected from: hydrogen, alkyl, cycloalkyl, C₁-C₁₂aryl, substituted alkyl, substituted cycloalkyl and substituted C₁-C₁₂aryl,
R¹¹ and R¹² are each independently selected from hydrogen, alkyl, substituted alkyl, C₃₋₆cycloalkyl, and aryl,
or R¹¹ and R¹² taken together with the nitrogen to which they are attached represent a 5 to 6 member saturated ring containing up to one other heteroatom selected from oxygen and nitrogen,
T is absent or selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
P is selected from OR¹⁰, SR¹⁰, NR¹¹R¹², and R¹⁰, where R¹⁰ is selected from: hydrogen, alkyl, cycloalkyl, C₁-C₁₂aryl, substituted alkyl, substituted cycloalkyl and substituted C₁-C₁₂aryl,
R²⁵ is selected from: hydrogen, alkyl, cycloalkyl, C₁-C₁₂aryl, substituted alkyl, substituted cycloalkyl and substituted C₁-C₁₂aryl,
R³⁰ is selected from: hydrogen, alkyl, halogen, cycloalkyl, C₁-C₁₂aryl, substituted alkyl, substituted cycloalkyl and substituted C₁-C₁₂aryl, and
R⁴⁵ is selected from: hydrogen, alkyl, halogen, cycloalkyl, C₁-C₁₂aryl, substituted alkyl, substituted cycloalkyl and substituted C₁-C₁₂aryl;
R⁵ is absent when the nitrogen attached thereto has a double bond or selected from the group consisting of: hydrogen, alkyl, cycloalkyl, C₁-C₁₂aryl, substituted alkyl, substituted cycloalkyl and substituted C₁-C₁₂aryl;
R⁶ is absent when the nitrogen attached thereto has a double bond or selected from the group consisting of: hydrogen, alkyl, cycloalkyl, C₁-C₁₂aryl, substituted alkyl, substituted cycloalkyl and substituted C₁-C₁₂aryl;
m is 0-6; and
AR is a cyclic or polycyclic aromatic ring containing from 3 to 16 carbon atoms, optionally containing one or more heteroatoms, provided that when the number of carbon atoms is 3 the aromatic ring contains at least two heteroatoms and when the number of carbon atoms is 4 the aromatic ring contains at least one heteroatom, optionally substituted with one or more substituents selected from the group consisting of: alkyl, substituted alkyl, cycloalkyl, substituted cycloalkyl, aryl, substituted aryl, aryloxy, hydroxy, alkoxy, acyloxy, —NR¹³R¹⁴, N-acylamino, N-sulfonylamino, nitro, cyano, halogen, —C(O)OR¹⁰, —C(O)NR¹³R¹⁴, —S(O)₂NR¹³R¹⁴, —S(O)ₙR¹⁰, protected —OH, and alkyl substituted with one or more substituents selected from the group consisting of: alkoxy, acyloxy, aryl, substituted aryl, amino, N-acylamino, oxo, hydroxy, cycloalkyl, substituted cycloalkyl, —C(O)OR¹⁰, —S(O)₂NR¹³R¹⁴, —S(O)ₙR¹⁰, aryloxy, nitro, cyano, halogen, and protected —OH,
where
n is 0 to 2;
R¹⁰ is selected from the group consisting of: hydrogen, alkyl, cycloalkyl, C₁-C₁₂aryl, substituted alkyl, substituted cycloalkyl and substituted C₁-C₁₂aryl, and
R¹² and R¹³ are independently selected from the group consisting of: hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, —NR¹⁰R¹⁰, N-acylamino, oxo, hydroxy, —C(O)OR¹⁰, —S(O)ₙR¹⁰, —C(O)NR¹¹R¹⁰, —S(O)₂NR¹⁰R¹⁰, nitro, cyano, cycloalkyl, substituted cycloalkyl, halogen, C₁-C₁₂aryl, substituted C₁-C₁₂aryl, and protected —OH,
where n and R¹⁰ are as described above;
and pharmaceutically acceptable salts, hydrates, solvates and esters thereof.

2. A compound of claim 1 represented by the following Formula (II): 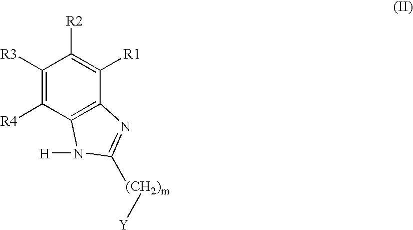
wherein:
R¹, R², R³ and R⁴ are each independently selected from hydrogen,
—(CH₂)ₚOR¹⁰, —C(O)OR¹⁰, formyl, nitro, cyano, halogen, aryl, substituted aryl, substituted alkyl, —S(O)ₙR¹⁰, cycloalkyl, —NR¹¹R¹², protected —OH,
—CONR¹¹R¹², phosphonic acid, sulfonic acid, phosphinic acid, —SO₂NR¹¹R¹², a heterocyclic methylene substituent as represented by Formula (III), 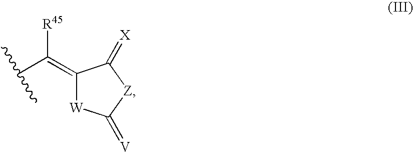
and
a substituent as represented by Formula (VII), 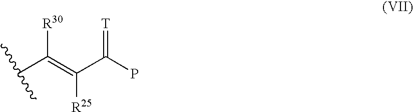
where,
p is 0-6,
n is 0-2,
W and Z are each independently selected from C, O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
V and X are each independently selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R¹⁰ is selected from: hydrogen, alkyl, cycloalkyl, C₁-C₁₂aryl, substituted alkyl, substituted cycloalkyl and substituted C₁-C₁₂aryl,
R¹¹ and R¹² are each independently selected from hydrogen, alkyl, substituted alkyl, C₃₋₆cycloalkyl, and aryl,
or R¹¹ and R¹² taken together with the nitrogen to which they are attached represent a 5 to 6 member saturated ring containing up to one other heteroatom selected from oxygen and nitrogen,
T is absent or selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
P is selected from OR¹⁰, SR¹⁰, NR¹¹R¹², and R¹⁰, where R¹⁰ is selected from: hydrogen, alkyl, cycloalkyl, C₁-C₁₂aryl, substituted alkyl, substituted cycloalkyl and substituted C₁-C₁₂aryl,
R²⁵ is selected from: hydrogen, alkyl, cycloalkyl, C₁-C₁₂aryl, substituted alkyl, substituted cycloalkyl and substituted C₁-C₁₂aryl,
R³⁰ is selected from: hydrogen, alkyl, cycloalkyl, C₁-C₁₂aryl, substituted alkyl, substituted cycloalkyl and substituted C₁-C₁₂aryl, and
R⁴⁵ is selected from: hydrogen, alkyl, halogen, cycloalkyl, C₁-C₁₂aryl, substituted alkyl, substituted cycloalkyl and substituted C₁-C₁₂aryl;
m is 0-6; and
Y is a cyclic or polycyclic aromatic ring containing from 4 to 14 carbon atoms, optionally containing from one to three heteroatoms, and optionally substituted with one or more substituents selected from the group consisting of: alkyl, substituted alkyl, C₁-C₁₂aryl, substituted cycloalkyl, substituted C₁-C₁₂aryl, hydroxy, aryloxy, alkoxy, cycloalkyl, nitro, cyano, halogen and protected —OH;
and pharmaceutically acceptable salts, hydrates, solvates and esters thereof.

3. A compound of claim 1 selected from:
5-{2-[6-(3,4-Dichloro-phenyl)-pyridin-2-yl]-1H-benzoimidazol-5-ylmethylene}-2-thioxo-thiozolidin-4-one;
5-{2-[6-(3,4-Dimethyl-phenyl)-pyridin-2-yl]-1H-benzoimidazol-5-ylmethylene}-2-thioxo-thiozolidin-4-one;
(E)-3-{2-[6-(4-tert-Butyl-phenyl)-pyridin-2-yl]-1H-benzoimidazol-5-yl}-2-methyl-acrylic acid;
5-(2-[5-(3,4-Dimethyl-phenyl)-thiophen-2-yl]-1H-benzoimidazol-5-ylmethylene}-2-thioxo-thiozolidin-4-one;
5-{2-[4-(3,4-Dimethyl-phenyl)-thiophen-2-yl]-1H-benzoimidazol-5-ylmethylene}-2-thioxo-thiozolidin-4-one;
5-{2-[5-(4-tert-Butyl-phenyl)-furan-2-yl]-1H-benzoimidazol-5-ylmethylene}-2-thioxo-thiozolidin-4-one;
5-[2-(4′-tert-Butyl-biphenyl-3yl)-1H-benzoimidazol-5-ylmethylene]-2-thioxo-thiozolidin-4-one;
and
5-[2-(4′-tert-Butyl-2-hydroxy-biphenyl-3yl)-1H-benzoimidazol-5-ylmethylene]-2-thioxo-thiozolidin-4-one;
and pharmaceutically acceptable salts, hydrates, solvates and esters thereof.

4. A method of treating of thrombocytopenia in a mammal, including a human, in need thereof which comprises administering to such mammal a therapeutically effective amount of a compound of Formula (I), as described in claim 1.

5. A method as claimed in claim 4, wherein the mammal is a human.

6. The method of claim 5 wherein the compound is selected from the compounds listed in claim 3.

7. A method of enhancing platelet production in a mammal, including a human, in need thereof which comprises administering to such mammal a therapeutically effective amount of a compound of claim 1.

8. A method as claimed in claim 7, wherein the mammal is a human.

9. The method of claim 8 wherein the compound is selected from the compounds listed in claim 3.

10. A pharmaceutical composition comprising a compound of claim 1 and a pharmaceutically acceptable carrier.

11-13. (canceled)

14. A method of agonizing the TPO receptor in a subject which comprises administering an effective amount of a compound of Formula (I), as described in claim 1.

15. A process for preparing a pharmaceutical composition containing a pharmaceutically acceptable carrier or diluent and an effective amount of a compound of the Formula (I) as described in claim 1 and pharmaceutically acceptable salts, hydrates, solvates and esters thereof which process comprises bringing the compound of the Formula (I) into association with the pharmaceutically acceptable carrier or diluent.

16. A method of claim 4 further comprising co-administering a therapeutically effective amount of an agent selected from the group consisting of: a colony stimulating factor, cytokine, chemokine, interleukin or cytokine receptor agonist or antagonists, soluble receptors, receptor agonists or antagonist antibodies, or small molecules or peptides that act by the same mechanisms one or more of said agents.

17-26. (canceled)

27. An in vitro or ex vivo method for enhancing the survival and/or proliferation of stem cells, bone marrow cells, cord-blood cells, peripheral blood cells or other types of cells expressing the TPO receptor in culture which comprises culturing said cell in a medium containing an effective amount of a compound of claim 1.

28. A method of claim 27 further comprising co-administration of a therapeutically effective amount of a colony stimulating factor, cytokine, chemokine, interleukin or cytokine receptor agonist.

29-43. (canceled)

44. An intermediate used in the preparation of compounds of claim 1 selected from:
N-[2-Nitro-4-(4-oxo-2-thioxo-thiazolidin-5-ylidenemethyl)-phenyl]-acetamide;
5-(4-Amino-3-nitro-benzylidene)-2-thioxo-thiazolidin-4-one;
5-(3,4-Diamino-benzylidene)-2-thioxo-thiazolidin-4-one;
(E)-3-(4-Acetylamino-3-nitro-phenyl)-2-methyl-acrylic acid ethyl ester;
(E)-3-(4-Amino-3-nitro-phenyl)-2-methyl-acrylic acid ethyl ester;
(E)-3-(3,4-Diamino-phenyl)-2-methyl-acrylic acid ethyl ester;
6-(3,4-Dimethyl-phenyl)-pyridine-2-carbaldehyde;
6-(3,4-Dichloro-phenyl)-pyridine-2-carbaldehyde;
6-(4-tert-Butyl-phenyl)-pyridine-2-carbaldehyde;
4′-tert-Butyl-2-methoxy-biphenyl-3-carbaldehyde; and
4′-tert-Butyl-2-hydroxy-biphenyl-3-carbaldehyde.
